(19) United States
(12) Patent Application Publication
(10) Pub. No.: US 2020/0201371 A1
(43) Pub. Date: Jun. 25, 2020

(54) LOW POWER CONSUMPTION INTEGRATING CIRCUIT BASED ON ADAPTIVE CURRENT REGULATION

(71) Applicant: CHONGQING PAIXINRUWEI TECH CO., LTD., Chongqing (CN)

(72) Inventor: Fang TANG, Chongqing (CN)

(73) Assignee: CHONGQING PAIXINRUWEI TECH CO., LTD., Chongqing (CN)

(21) Appl. No.: 16/705,252

(22) Filed: Dec. 6, 2019

(30) Foreign Application Priority Data
Dec. 25, 2018 (CN) 201811588424.9

(51) Int. Cl. G05F 1/46; H03F 3/213

(52) U.S. Cl. CPC G05F 1/46; A61B 5/14557; H03F 3/213

(57) ABSTRACT

A low power consumption integrating circuit based on adaptive current regulation, including an amplifier A2, a capacitor Ci, a bias current regulating circuit, where a negative power source terminal of the amplifier A2 is connected to an output terminal of the amplifier A2 through the capacitor Ci, an output of the bias current regulating circuit is connected to an input terminal of the amplifier A2; and the bias current regulating circuit adjusts a bias current according to different light intensity. According to the present invention, the bias current regulating circuit dynamically adjusts, according to light intensity, a bias current input to the amplifier A2, so as to significantly reduce the overall power consumption of the circuit while ensuring a rapid response capability of an optical frequency sensor.

### CROSS REFERENCE TO THE RELATED APPLICATIONS

This application is based upon and claims priority to Chinese Patent Application No. 201811588424.9, filed on Dec. 25, 2018, the entire contents of which are incorporated herein by reference.

### TECHNICAL FIELD

The present invention belongs to the integrated circuit design field, and more specifically, relates to a low power consumption integrating circuit based on adaptive current regulation.

### BACKGROUND

An integrating circuit is an important link to implement signal restoration, and is a core part of an optical frequency sensor. The integrating circuit is mainly used for converting a photocurrent generated by a photodiode into a voltage signal. The performance of the integrating circuit determines a signal spectrum range and signal response time, and directly affects the steady state performance and transient performance of the optical frequency sensor.

In the field of optical frequency sensor chips in blood oxygen detectors, portable medical equipment is developing towards low power consumption equipment. In an optical frequency sensor chip, the power consumption of an integrating circuit occupies a relatively high proportion. To make an output frequency to rapidly keep up with a change in input light intensity, an operation amplifier needs to have relatively high bandwidth, to ensure that a loop has a relatively high phase margin, otherwise, pulsed-light-responsive damped oscillation of the output frequency occurs, significantly increasing time used for establishing the output frequency, and finally leading to a failure of a blood oxygen saturation detection system. Therefore, a fast response integrating circuit has relatively high power consumption. Especially, when a conventional integrating circuit is used, because a bias current of an operation amplifier of the integrating circuit is constant, current consumption of a closed loop amplifier needs to meet a phase margin required when a maximum output frequency is used, and cannot dynamically adjust the power consumption of the operation amplifier to match different light intensity requirements.

### SUMMARY

The present invention aims to resolve at least one technical problem in a related technology to some extent. Therefore, a main objective of the present invention is to provide a low power consumption integrating circuit based on adaptive current regulation, so as to resolve a problem that a conventional integrating circuit cannot dynamically adjust the power consumption of an operation amplifier to match different light intensity requirements.

The objective of the present invention is implemented through the following technical solution:

A low power consumption integrating circuit based on adaptive current regulation, including an amplifier A2, a capacitor Ci, and a bias current regulating circuit, where

a negative power source terminal of the amplifier A2 is connected to an output terminal of the amplifier A2 through the capacitor Ci;

an output of the bias current regulating circuit is connected to an input terminal of the amplifier A2; and

the bias current regulating circuit adjusts a bias current according to different light intensity.

Further, the bias current regulating circuit includes a light intensity positively-correlated control voltage generating circuit and a bias current generating circuit;

a control voltage generated by the light intensity positively-correlated control voltage generating circuit is used for dynamically adjusting a current output of the bias current generating circuit; and

the bias current generating circuit generates a corresponding output current according to the control voltage, and outputs the current to the input terminal of the amplifier A2, to adjust the power consumption of A2.

Further, the control voltage generated by the light intensity positively-correlated control voltage generating circuit is positively correlated with a photocurrent.

Further, the control voltage generated by the light intensity positively-correlated control voltage generating circuit is obtained from a low leakage photocurrent buffer.

Compared with the prior art, the present invention has at least the following advantages: the power consumption of an operation amplifier can be dynamically adjusted according to different light intensity while a rapid response capability is ensured, so as to reduce the overall power consumption.

### BRIEF DESCRIPTION OF THE DRAWINGS

To describe the technical solutions in the embodiments of the present invention or in the prior art more clearly, the following briefly describes the accompanying drawings required for describing the embodiments or the prior art. Apparently, the accompanying drawings in the following description show some embodiments of the present invention, and a person of ordinary skill in the art may still derive other drawings from these accompanying drawings without creative efforts.

FIG. 1 shows a structural schematic diagram of a low power consumption integrating circuit based on adaptive current regulation;

FIG. 2 shows a structural schematic diagram of a light intensity positively-correlated control voltage generating circuit;

FIG. 3 shows a structural schematic diagram of a bias current generating circuit;

FIG. 4 shows a schematic diagram of a correlation between a control voltage VC and a photocurrent; and

FIG. 5 shows a schematic diagram of a correlation between a control voltage VC and a bias current IbA of an amplifier.

### DETAILED DESCRIPTION OF THE EMBODIMENTS

The following clearly and completely describes the technical solutions in the embodiments of the present invention with reference to the accompanying drawings in the embodiments of the present invention. Apparently, the described embodiments are merely a part rather than all of the embodiments of the present invention.

All other embodiments obtained by a person of ordinary skill in the art based on the embodiments of the present invention without creative efforts shall fall within the protection scope of the present invention. All other embodiments obtained by a person of ordinary skill in the art based on the embodiments of the present invention without creative efforts shall fall within the protection scope of the present invention.

It should be noted that all the directional indications (such as upper, lower, left, right, front, and back) in the embodiments of the present invention are merely used to explain a relative position relationship, motion situations, and the like of the components in a specific gesture (as shown in the figures). If the specific gesture changes, the directivity indication also changes accordingly.

Moreover, the terms such as “first”, “second”, and the like described in the present invention are used herein only for the purpose of description and are not intended to indicate or imply relative importance, or implicitly indicate the number of the indicated technical features. Therefore, features defined by “first” and “second” may explicitly or implicitly include at least one of the features.

In description of the present invention, “a plurality of” means at least two, for example, two or three, unless otherwise clearly and specifically limited.

In the present invention, unless otherwise clearly specified and limited, meanings of terms “connection”, “fastening”, and the like should be understood in a board sense. For example, “connection” may be a fixed connection, a removable connection, or integration; may be a mechanical connection or an electrical connection; may be a direct connection or an indirect connection implemented by using an intermediate medium; or may be intercommunication between two components or an interaction relationship between two components, unless otherwise clearly limited. A person of ordinary skill in the art may understand specific meanings of the foregoing terms in the present invention based on a specific situation.

Furthermore, the technical solutions between the various embodiments of the present invention may be combined with each other, but must be on the basis that the combination thereof can be implemented by a person of ordinary skill in the art. In case of a contradiction with the combination of the technical solutions or a failure to implement the combination, it should be considered that the combination of the technical solutions does not exist, and is not within the protection scope of the present invention.

### Embodiment 1

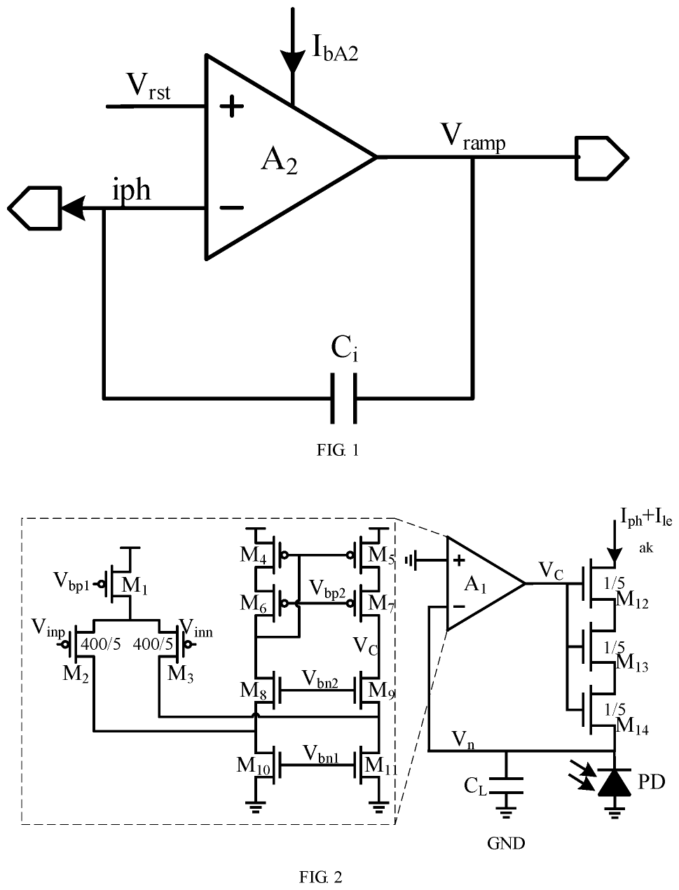

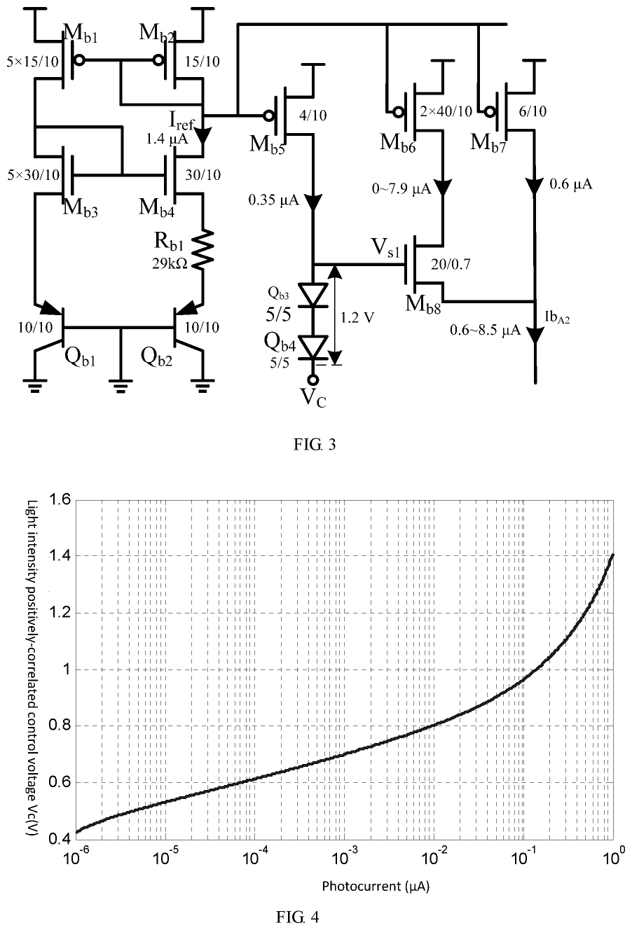

The embodiment of the present invention provides a low power consumption integrating circuit based on adaptive current regulation, as shown in FIG. 1, FIG. 2, and FIG. 3.

The low power consumption integrating circuit based on adaptive current regulation includes an amplifier A2, a capacitor Ci, and a bias current regulating circuit.

A negative power source terminal of the amplifier A2 is connected to an output terminal of the amplifier A2 through the capacitor Ci.

An output of the bias current regulating circuit is connected to an input terminal of the amplifier A2.

The bias current regulating circuit includes a light intensity positively-correlated control voltage generating circuit and a bias current generating circuit.

The light intensity positively-correlated control voltage generating circuit includes a standard folding common-gate differential amplifier A1, a capacitor CL, MOS transistors M12 to M14, and a photosensitive diode PD.

A positive power source terminal of A1 is grounded, a negative power source terminal thereof is connected to both an electrode of CL and a negative electrode of PD, and the other electrode of CL is grounded; and all gate electrodes of M12 to M14 are connected to an output terminal of A1.

All the gate electrodes of M12 to M14 are connected to the output terminal of A1, a drain electrode of M12 is connected to Iₚₕ and Iₗₑₐₖ, a source electrode of M12 is connected to a drain electrode of M13, a source electrode of M13 is connected to a drain electrode of M14, a source electrode of M14 is connected to the negative electrode of PD, and a positive electrode of PD is grounded.

A voltage of the drain electrode of M12 is 300 mV by default, and M12 to M14 all are 5V-type transistors.

The bias current generating circuit includes MOS transistors Mb1 to Mb8, triodes Qb1 and Qb2, diodes Qb3 and Qb4, and a resistor Rb1.

A source electrode of Mb1 is connected to a drain electrode of Mb3, a source electrode of Mb3 is connected to an emitting electrode of Qb1, and a collector electrode of Qb1 is grounded.

A source electrode of Mb2 is connected to a drain electrode of Mb4, a source electrode of Mb4 is connected to an emitting electrode of Qb2 through Rb1, and a collector electrode of Qb2 is grounded.

A gate electrode of Mb1 is connected to a gate electrode of Mb2 and the source electrode of Mb2.

The source electrode of Mb1 is connected to a gate electrode of Mb3.

A base electrode of Qb1 and a base electrode of Qb2 are connected and then grounded.

The source electrode of Mb2 is connected to a gate electrode of Mb5, a gate electrode of Mb6, and a gate electrode of Mb7.

A positive electrode of Qb3 is connected to both a source electrode of Mb5 and a gate electrode of Mb8, a negative electrode of Qb3 is connected to a positive electrode of Qb4, and the positive electrode of Qb4 is connected to Vc.

A source electrode of Mb6 is connected to a drain electrode of Mb8, and a source electrode of Mb8 is connected to a source electrode of Mb7.

The gate electrode of Mb8 is connected to the positive electrode of Qb3.

A voltage of the positive electrode of Qb3 is 1.2V greater than a voltage Vc.

In the present invention, M12 to M14 may be approximate to a lumped long channel NMOS transistor ML in a saturation region, a control voltage VC may be expressed as Formula 1, where V_{th_ML} and β_{n_ML} are effective coefficients of the transistor ML. A simulation result shown in FIG. 4 shows that the control voltage VC is positively correlated with a photocurrent, and may be used for a proposed LIPC control voltage.

$${V}_{C} = \sqrt{\frac{2 ( {I}_{ph} + {I}_{leak} )}{{β}_{n \_ ML}}} + {V}_{th \_ ML} \qquad Formula 1$$

In a bias control generator circuit, a constant current 600 nA is absorbed to a light intensity positively-correlated control voltage VC by stacking diodes Qb3 and Qb4 are stacked. A control voltage Vs1 can be generated at a fixed voltage that is 1.2V higher than VC.

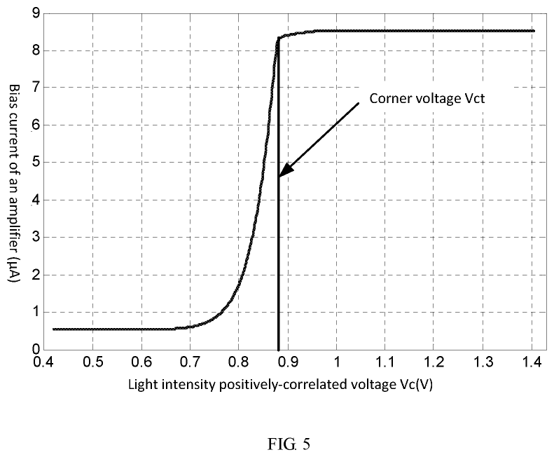

FIG. 5 shows a relationship between a control voltage VC and a bias current IbA of an amplifier. When VC is higher than 0.9 V, a NMOS transistor Mb8 is in a linear region, and can be regarded as a conduction switch. If VC is lower than a corner voltage VCt, Mb8 enters a saturation region, and then Mb6 is in the linear region. As VC continuously decreases, Mb8 is switched off because of underactuation. Finally, an output bias current only keeps at 0.6 μA, and the current is entirely led out through a current mirror transistor Mb7.

The total power consumption of the whole circuit is mainly determined by the amplifier A2. Therefore, the overall power consumption of the circuit can be significantly reduced by dynamically adjusting a bias current of the amplifier A2 according to a requirement.

The above merely describes specific embodiments of the present invention, but the protection scope of the present invention is not limited thereto. A person skilled in the art can easily conceive modifications or replacements within the technical scope of the present invention, and these modifications or replacements shall fall within the protection scope of the present invention. Therefore, the protection scope of the present invention should be subject to the protection scope of the claims.

What is claimed is:

1. A low power consumption integrating circuit based on adaptive current regulation, comprising an amplifier A2, a capacitor Ci, and a bias current regulating circuit, wherein
a negative power source terminal of the amplifier A2 is connected to an output terminal of the amplifier A2 through the capacitor Ci;
an output of the bias current regulating circuit is connected to an input terminal of the amplifier A2; and
the bias current regulating circuit adjusts a bias current according to different light intensity.

2. The circuit according to claim 1, wherein the bias current regulating circuit comprises a light intensity positively-correlated control voltage generating circuit and a bias current generating circuit;
a control voltage generated by the light intensity positively-correlated control voltage generating circuit is used for dynamically adjusting a current output of the bias current generating circuit; and
the bias current generating circuit generates a corresponding output current according to the control voltage, and outputs the corresponding output current to the input terminal of the amplifier A2, to adjust a power consumption of A2.

3. The circuit according to claim 2, wherein the control voltage generated by the light intensity positively-correlated control voltage generating circuit is positively correlated with a photocurrent.

4. The circuit according to claim 2, wherein the control voltage generated by the light intensity positively-correlated control voltage generating circuit is obtained from a low leakage photocurrent buffer.
